(19) United States
(12) Patent Application Publication
(10) Pub. No.: US 2012/0059310 A1
(43) Pub. Date: Mar. 8, 2012

(54) VIBRATION INDUCED IN VIVO SUBSTANCE TARGETING

(76) Inventor: Rao Papineni, Branford, CT (US)

(21) Appl. No.: 13/226,807

(22) Filed: Sep. 7, 2011

Related U.S. Application Data
(60) Provisional application No. 61/402,897, filed on Sep. 7, 2010.

(51) Int. Cl. A61M 37/00

(52) U.S. Cl. 604/22

(57) ABSTRACT

A method for aggregating a substance in a localized region within a body of an animal comprises applying a vibration pattern that causes aggregation of the substance to the localized region within the body of the animal, introducing the substance and aggregating the substance in the localized region based upon application of the vibration pattern. The substance, which may be a drug, fluorescent dye, conjugate of the two or a smart probe, may aggregate to bone, specifically to the lumbar region of the spine. About two to about four times more substance may aggregate in the localized region of the body relative to a control where no vibration pattern is applied.

### CROSS-REFERENCE TO RELATED APPLICATION

This application claims priority to U.S. Provisional Application Ser. No. 61/402,897, filed on Sep. 7, 2010, which is incorporated herein in its entirety.

### FIELD OF THE INVENTION

The present invention relates to methods for aggregating substances within localized regions of a body of an animal and more particularly to enhanced aggregation of substances to bone.

### BACKGROUND OF THE INVENTION

In order to increase the efficiency of therapeutic drugs, the possibility of targeting drugs and other substances to specific areas of the body is being explored. The targeting of drugs to a desired site within the body will ameliorate the problem of drug toxicity by lowering the drug-doses prescribed and also concentrating drugs in a localized manner, and increasing the dwell time of the drugs at the site of interest.

Among those drugs whose efficacy would be improved by site-specific targeting are Bisphosphonates. Bisphosphonates are potent inhibitors of bone resorption that also provide pain relief and improve bone health. However, use of this class of drugs in high intravenous doses results in complications such as bisphosphonate-related osteonecrosis of the jaw (BRONJ) and subtrochanteric atypical fractures. Although these complications generally arise in patients being treated for cancer or trauma, they have also been reported in patients being treated for osteoporosis. Thus, site-selective skeletal targeting of smaller doses of Bisphosphonates is desirable. While previous drug targeting systems may have achieved certain degrees of success in their particular applications, there remains a need for improved targeting systems.

### SUMMARY OF THE INVENTION

In one embodiment, the present invention comprises a method for aggregating a substance in a localized region within a body of an animal comprising applying a vibration pattern that causes aggregation of the substance to the localized region within the body of the animal, introducing the substance into the body of the animal and aggregating the substance in the localized region based upon application of the vibration pattern.

In another embodiment, the present invention comprises a method of aggregating a substance to the lumbar region of the spine within a body of an animal. The method comprises applying a vibration pattern that causes aggregation of the substance to that region.

In still another embodiment, the present invention comprises a method of aggregating a substance in a localized region of the body based upon a vibration pattern, wherein between about two to about four times more substance aggregates in the localized region of the body relative to a control where no vibration pattern is applied.

The foregoing embodiments can incorporate various additional features and steps. The aggregated substance may be visualized non-invasively through conjugation of the substance to a fluorescent dye. The vibration frequency applied during vibration may be up to about 100 Hz, preferably between about 10 and about 100 Hz, more preferably between about 35 and about 75 Hz and the vibration pattern may be applied multiple times in a predetermined pattern (e.g., for at least 5 consecutive days) prior to introducing the substance. The vibration pattern may be applied to a localized point on the body of the animal and the aggregation may occur in the lumbar vertebrae. The substance may be a drug useful for treating a disease state and that aggregates to bone, such as Bisphosphonate.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and other objects, features, and advantages of the invention will be apparent from the following more particular description of the embodiments of the invention, as illustrated in the accompanying figures. The elements of the figures are not necessarily to scale relative to each other.

FIG. 1 shows mice treated with a whole body vibration pattern prior to administration of LSS-Alendronate.

FIG. 2 shows mice treated with a localized vibration protocol prior to administration of Osteosense 750.

FIG. 3A shows immunohistochemical staining of the lumbar vertebrae of a mouse subjected to multiple exposures of vibration.

FIG. 3B shows immunohistochemical staining of muscle sections of mice subjected to a single exposure or multiple exposures of vibration.

### DETAILED DESCRIPTION OF THE INVENTION

The present invention relates to a method for aggregating a substance in a localized region within a body of an animal. Various vibration patterns may be applied and the animal may be a laboratory animal under study, such as a mouse, chimpanzee or others. The methods herein may, however, also be applied to humans. Typically, between about two to about four times more substance aggregates in the localized region of the body of the animal relative to a control where no vibration pattern is applied. The vibration may also increase the residence time of the substance in the localized region within the animal's body.

Vibration suitable for use in the present invention may be whole body vibration (WBV) or vibration localized to one region of the body (localized vibration). Methods and machines useful for applying vibration to the body of an animal are known. For example, a whole body vibration machine commercially available from Health Mark of (Denver, Colo.) may be employed to supply the necessary vibration. Localized vibration may be applied by an Acuvibe HT-1230 mini handheld vibrator. A range of vibration frequencies are also suitable for use in the current invention, including a vibration frequency from about 35 to about 75 Hz.

Further, a variety of vibration application or loading regimens may be used in the present invention. Parameters of the vibration regimen that can be varied include the duration of time each vibration treatment is applied to the animal, the number of vibration treatments the animal is subjected to, and the duration of the treatment regimen. The vibration may, for example, be applied multiple times in a predetermined pattern prior to introducing the substance. Preferably, the vibration pattern is applied once per day for between two and ten days, and preferably for five days.

The vibration pattern and the location of the vibration on the body can control where aggregation occurs. WBV or localized vibration on the body of the animal may be employed. In Example 3 below, a vibration pattern applied to a localized point on the right flank of the animal causes the substance to aggregate in the lumbar vertebrae.

The term substance, as used herein, means any substance capable of introduction into the body of an animal and capable of aggregating to localized regions of the animal's body. Substances suitable for use in the present invention include drugs, encapsulated drugs, dyes, drug-dye conjugates, molecular probes, and activatable smart probes.

Drugs useful in the present invention include but are not limited to Bisphosphonates, such as alendronate sodium, risedronate, pamidronate disodium, zoledronic acid, medronate, oxidronate, bisphosphonate coated or conjugated antibiotics, and anabolic drugs.

Suitable dyes include fluorescent dyes that fluoresce in a range from 400 to 1000 nm. Classes of dyes include, but are not limited to oxonol, pyrylium, Squaric, croconic, rodizonic, polyazaindacenes or coumarins, scintillation dyes (usually oxazoles and oxadiazoles), aryl- and heteroaryl-substituted polyolefins (C₂-C₈ olefin portion), merocyanines, carbocyanines, phthalocyanines, oxazines, carbostyryl, porphyrin dyes, dipyrrometheneboron difluoride dyes aza-dipyrrometheneboron difluoride dyes, and oxazine dyes. Commercially available fluorescent dyes useful in the invention are described in U.S. Pat. Appl. No. 2008/0181965. Additional fluorescent dyes useful in this invention include large stokes shift dyes described in U.S. Pat. Appl. No. 2008/0206886. The relevant portions of the disclosures of these published applications are incorporated by reference herein.

In certain embodiments, the substance used in this invention may be a drug conjugated to a fluorescent dye. Examples include alendronate sodium conjugated to a large stokes shift dye 640 shown below, or pamindronate conjugated to a large stokes shift dye 640.

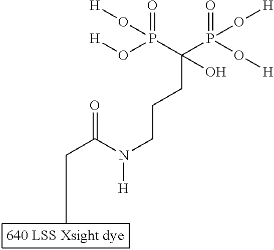

Other suitable drug-dye conjugates include Osteosense™ probe and Osteosense™ 750 by PerkinElmer.

Additional substances useful in the present invention include activatable molecular multimodal imaging probes also known as “smart probes” or “activatable probes.” A variety of smart probes are known in the art including those described in U.S. Pat. Appl. Nos. 2009/0098057, 2010/0233085, and U.S. Pat. No. 7,790,392. Activatable probes are rapidly becoming a smart and essential tool in optical imaging of in vivo molecular targets. Smart probes may be employed and guided through vibration to sites of inflammation and other sites of interest to obtain diagnostic data and in some cases theragnostics (combination therapeutic and diagnostic). To that end, the smart probe may be conjugated to a drug.

One suitable smart probe is the activatable molecular multimodal imaging probe that can be synthesized by conjugating biotin to a water soluble, near-infrared (NIR) tricarbocyanine, cyclic enamine-functionalized dye. After conjugation, the biotin-dye conjugate is incubated with neutravidin PEGylated gold nanoparticles. The high biotin-binding affinity of this fluorescence activatable system can be exploited by incorporating novel peptide substrates to the biotin-dye complex. For example, the quenched nanoparticles can be linked, by a linker moiety, to a substrate that is responsive to enzymes such as cathepsin K or a matrix metalloproteinase. Peptide substrates that recognize Cathepsin K include His-Pro-Gly-Gly-Pro-Gln. A dye or dye conjugate can be linked to the same substrate directly by peptide conjugation. Upon cleavage of the substrate by the enzyme, the dye or dye conjugate is cleaved from the nanoparticle, also relieving the quenching property.

The resultant quenching and unquenching processes of the neutravidin PEGylated gold nanoparticle and biotin-dye conjugated substrates can be monitored in vivo by NIR fluorescence, computed tomography CT and planar bench top X-ray modalities. In a further embodiment, to enhance the selectivity of the activatable probe, a biotinylated targeting macromolecule can be linked along with the substrate-dye complex. The nanoparticles directed by vibrations can be utilized for enhancing the radiation killing capacity as it is now known that gold nanoparticles act as radio-sensitizers.

The aggregated substance can also be visualized non-invasively through cell imaging systems, specific tissue imaging systems, drug delivery systems, etc. A suitable system for fluorescent detection by imaging, i.e., detection at multiple, spatially distributed points, is a Carestream In-Vivo Imaging System FX Pro, also commercially available from Carestream Health, Inc.

The method for aggregating a substance in a localized region within a body of an animal may further comprise diagnosing and/or treating a disease state. The disease state may be diagnosed/treated by increased or decreased aggregation of the substance. The disease state may be at least one of osteoporosis, osteoarthritis, corticosteroid-induced osteoporosis, Paget's disease, rheumatoid arthritis. bone metastasis (with or without hypercalcaemia), multiple myeloma, primary hyperparathyroidism, osteogenesis imperfecta, and other conditions that feature bone fragility. In an embodiment, the disease state is an infection. In another embodiment, the disease state is a deep infection at the lumbar region in an elderly patient. Treatment can involve trafficking stem cells in stem cell therapy or delivery of chemicals to tumors.

The following examples, illustrate, but are not intended to limit the scope of the claimed invention. The values expressed in the Tables within the Examples are in arbitrary units (A.U.).

### Example 1

### Preparation of the Bisphosphonate-Near-IR Dye Conjugate

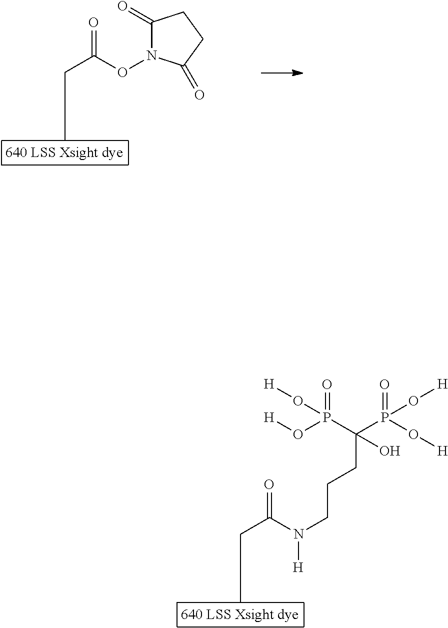

A solution containing 5.57 mg (5.0 μmol) of LSS dye (near-infrared tricarbocyanine, cyclic enamine functionalized dye) as its NHS ester in 1.0 ml of PBS buffer at pH 8.7 and 0.5 ml of THF was treated with 35.2 g (100 μmol) of alendronate and 25.9 (200 μmol) of diisopropylethylamine, then stirred at ambient temperature overnight. The resultant product was purified directly by preparative HPLC using a 20 mm×20 cm Phenomenex Luna, 10 μm C-18 with a gradient over 15 minutes of 10-50% methanol versus 0.1 M ammonium acetate buffer at pH 6.9. The resulting product was lyophilized three times to yield 2.4 mg of the product as a light, fluffy dark blue-green powder, 99% purity by HPLC-MS with a consistent mass spectra. The excitation spectra λ max observed=642 nm, emission spectra λ max=740, thus displaying a large Stokes shift.

### Example 2

### Whole Body Vibration

Mice were places in plastic cages deep enough to prevent escape. These plastic cages were strapped onto the bottom platform of a whole body vibration (WBV) machine commercially available from Health Mark (Denver, Colo.) that was used without the upper support bar and handles. Two sets of adult mice, each set containing three mice, were studied. All the adult mice used for sets were NMRI-Foxn1nu mice.

The first set of mice was a control, with no vibration applied. The second set of mice was exposed to a multiple vibration pattern—between 5 to 10 times/week for duration of 21 days.

The bisphosphonate-Near-IR dye conjugate from Example 1 (LSS-Alendronate) was injected through the tail-vein (i.v.) at the end of the vibration regime for each set of mice. Fluorescence monitoring was performed 24 hours after administration of the payload.

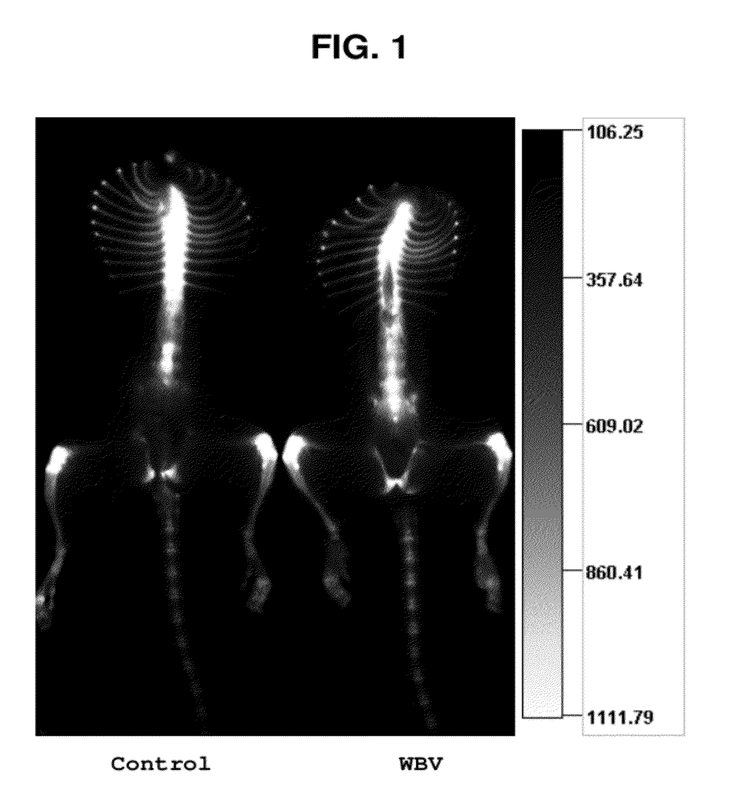

The results shown in Table 1 and FIG. 1 demonstrate aggregation of the injected substance at the lumbar vertebrae.

TABLE 1

| | | Net | Mean |
|---|---|---|---|
| Subject | Region | Intensity | Intensity |
| Control | Left limb | 3036817 | 706.46 |
| Control | Right limb | 3864810 | 844.47 |
| WBV | Left limb | 3806287 | 882.4 |
| WBV | Right limb | 3907118 | 889.71 |
| Control | Lumbar | 686490 | 491.05 |
| WBV | Lumbar | 1550279 | 894.1 |

### Example 3

### Localized Vibration

To provide a localized vibration, an Acuvibe HT-1230 mini handheld vibrator was used. Three sets of mice, each set containing three mice, were studied. As with Example 2, all adult mice used were NMRI-Foxn1nu mice.

The first set of mice served as a control, with no vibration applied. The second set was exposed to a single vibration event at a vibration frequency of 70 Hz for five minutes. The third set of mice was exposed to a multiple vibration pattern—utilizing a vibration frequency of 70 Hz for five minutes, 5 times/week at the right thigh region for 21 days.

Osteosense 750 from PerkinElmer was injected through the tail-vein (i.v.) at the end of the vibration regime for each set of mice. Monitoring fluorescence was performed 24 hours after administration of the payload.

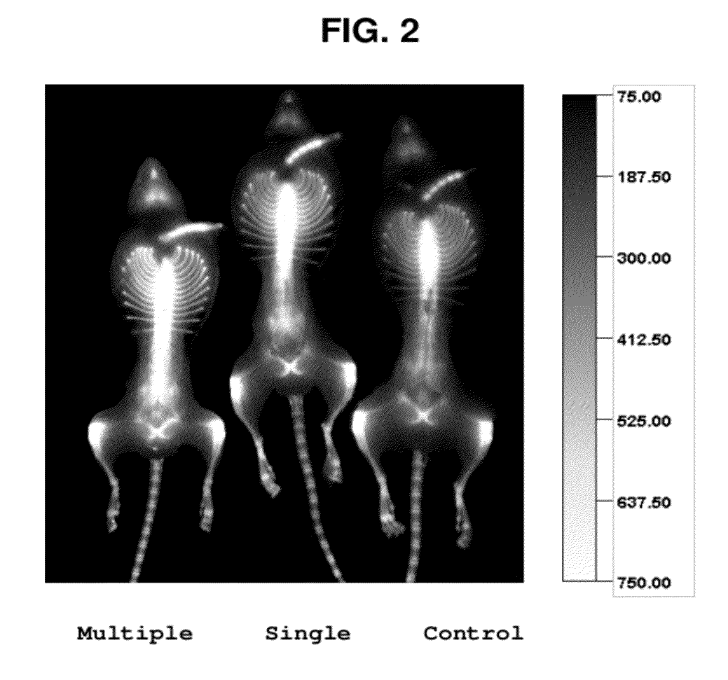

The results in Table 2 and FIG. 2 demonstrate enhanced aggregation of Osteosense 750 to the ventral surface of the lumbar vertebrae through localized vibration loading.

TABLE 2
Net Intensity Ratio

| | Multiple | Single | |
|---|---|---|---|
| Region | Vibration | Vibration | Control |
| Lumbar | 2.56 | 1.75 | 1 |
| Knee | 1.06 | 0.68 | 1 |
| NT-Thigh | 1.22 | 1.1 | 1 |

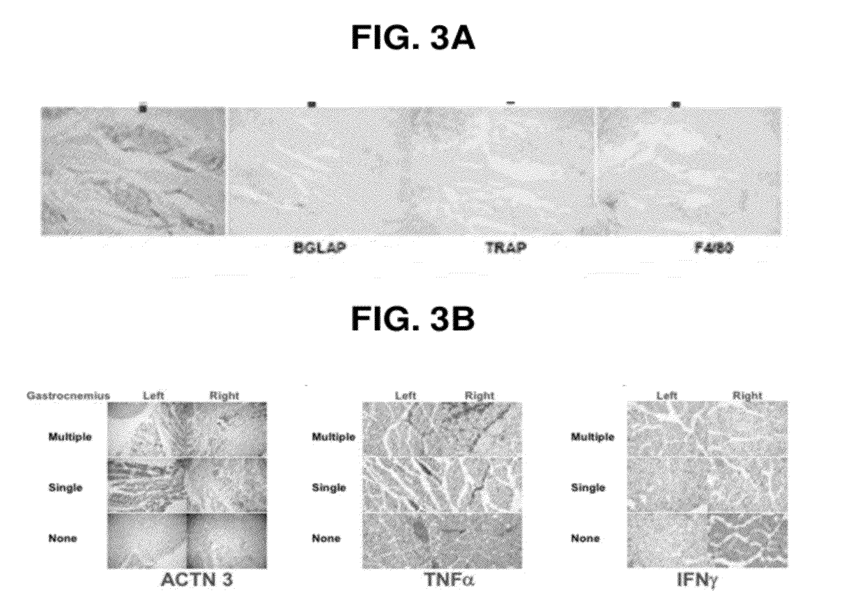

In order to confirm that the increased aggregation of the substance was due to the vibration and not increased inflammation, histopathology studies were also conducted. The whole backbone was decalcified and sectioned for histopathology. Specifically, sections of the lumbar regions were stained with tartrate-resistant acid phosphatase (TRAP) for staining of osteoclasts, osteocalcin bone gamma carboxyglutamic acid containing protein (BGLAP) for osteoblasts and F4/80 for the F4/80 glycoprotein expressed on murine macrophages. In addition, the gastrocnemius muscle was sectioned and stained with 3,3′ diaminobenzidine (DAB) with horse radish peroxidase and hemotoxylin. As shown in FIGS. 3A (spinal sections) and 3B (gastrocnemius sections), these studies rule out the possibility of any muscular or bone morphological changes.

### Example 4

### (Prophetic) Diagnosis of Early Stages of Rheumatoid Arthritis

Localized vibration will be applied to an animal, preferably a human to detect early stages of Rheumatoid Arthritis (“RA”). The vibration pattern will be applied in a predetermined pattern and allows changes in the perfusion at the synovial joints. The changes will be detected by imaging the endogenous fluorescence of the microcirculation. For confirmation of early onset of RA, a bolus of ICG (indocyanine dye) will be introduced by to obtain near-infrared imaging. The animal or body part may be imaged with a commercially available imaging system—bench-top multimodal (X-ray and optical) imaging apparatus, such as the Carestream in-Vivo Multispectral FX Pro.

The invention has been described in detail with particular reference to presently preferred embodiments, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. A method for aggregating a substance in a localized region within a body of an animal, comprising:
applying a vibration pattern that causes aggregation of the substance to the localized region within the body of the animal;
introducing the substance into the body of the animal; and
aggregating the substance in the localized region based upon application of the vibration pattern.

2. The method of claim 1, wherein the aggregated substance is visualized non-invasively through conjugation of the substance to a fluorescent dye.

3. The method of claim 1, wherein a vibration frequency between about 10 and about 100 Hz is applied during the applying step.

4. The method of claim 1, wherein the vibration pattern is applied to a localized point on the body of the animal and the aggregation is to the lumbar vertebrae.

5. The method of claim 1, wherein the substance is a drug that aggregates to bone.

6. The method of claim 5, wherein the drug is a bisphosphonate and further comprising treating a disease state with the bisphosphonate.

7. The method of claim 1, wherein the vibration pattern is applied multiple times in a predetermined pattern prior to introducing the substance.

8. The method of claim 7, wherein the vibration pattern is applied for at least 6 consecutive days prior to introducing the substance.

9. The method of claim 1, wherein between about two to about four times more substance aggregates in the localized region of the body relative to a control where no vibration pattern is applied.

10. A method of aggregating a substance in a localized region within a body of an animal, comprising:
applying a vibration pattern that causes aggregation of the substance to the localized region within the body of the animal, wherein the localized region is the lumbar region;
subjecting the body of the animal to the vibration pattern;
introducing the substance into the body of the animal; and
aggregating the substance in the lumbar region based upon the vibration pattern.

11. The method of claim 10, wherein the aggregated substance is visualized non-invasively through conjugation of the substance to a fluorescent dye.

12. The method of claim 10, wherein the vibration frequency is between about 10 and about 100 Hz.

13. The method of claim 10, wherein the substance is a drug that aggregates to bone.

14. The method of claim 13, further comprising treating a disease state with the aggregated drug.

15. The method of claim 14, wherein the disease state is at least one of osteoporosis, osteoarthritis, rheumatoid arthritis, multiple myeloma, primary hyperparathyroidism, osteogenesis imperfecta and Paget's disease.

16. The method of claim 10, wherein the vibration is applied multiple times in a predetermined pattern prior to introducing the substance.

17. The method of claim 10, wherein between about two to about four times more substance aggregates in the localized region of the body relative to a control where no vibration pattern is applied.

18. A method of aggregating a substance in a localized region within a body of an animal, comprising:
applying a vibration pattern that causes aggregation of the substance to the localized region within the body of the animal;
introducing the substance into the body of the animal; and
aggregating the substance in the localized region based upon the vibration pattern, wherein between about two to about four times more substance aggregates in the localized region of the body relative to a control where no vibration pattern is applied.

19. The method of claim 18, wherein the substance is a drug and further comprising treating a disease state with the aggregated drug.

20. The method of claim 18, wherein the vibration pattern is applied to a localized point on the body of the animal.
